(19) United States
(12) Patent Application Publication
(10) Pub. No.: US 2026/0148349 A1
(43) Pub. Date: May 28, 2026

(54) SYSTEMS AND METHODS FOR GENERATING CONTRAST-ENHANCED MAGNETIC RESONANCE IMAGES

(71) Applicants: Case Western Reserve University, Cleveland, OH (US); University Hospitals Cleveland Medical Center, Cleveland, OH (US)

(72) Inventors: Eunate Alzaga Goñi, Cleveland, OH (US); Sree Gongala, Cleveland, OH (US); Walter Zhao, Cleveland, OH (US); Dan Ma, Cleveland, OH (US); Chaitra Badve, Cleveland, OH (US)

(21) Appl. No.: 19/402,718

(22) Filed: Nov. 26, 2025

Related U.S. Application Data
(60) Provisional application No. 63/725,277, filed on Nov. 26, 2024.

(51) Int. Cl. G06T 5/60; A61B 5/00; A61B 5/055; G01R 33/50; G01R 33/56

(52) U.S. Cl. CPC G06T 5/60; A61B 5/055; A61B 5/742; G01R 33/50; G01R 33/5608; G06T 2207/10088; G06T 2207/20081; G06T 2207/20084

(57) ABSTRACT

A system for generating contrast-enhanced magnetic resonance images of a subject includes an input configured to receive at least one non-contrast enhanced image of the subject, and a contrast-enhanced magnetic resonance (MR) image synthesis neural network coupled to the input and configured to generate a contrast-enhanced magnetic resonance image of the subject based on the at least one non-contrast enhanced image of the subject. The contrast-enhanced MR image synthesis neural network is trained using a set of training data comprising at least quantitative data.

### CROSS-REFERENCE TO RELATED APPLICATIONS

This application is based on, claims priority to, and incorporates herein by reference in its entirety U.S. Ser. No. 63/725,277 filed Nov. 26, 2024, and entitled “Deep-Learning-Based Synthesis of Gadolinium-Enhanced MRI From Non-Contrast MRI and MR Fingerprinting”.

### STATEMENT REGARDING FEDERALLY SPONSORED RESEARCH

This invention was made with government support under CA269604 awarded by the National Institutes of Health. The government has certain rights in the invention.

### BACKGROUND

Contrast-enhanced magnetic resonance imaging (MRI) is an imaging technique that utilizes images acquired after the intravenous administration of a gadolinium-based contrast agent (GBCA). In standard contrast-enhanced MRI (CE-MRI), images are acquired both before and after the administration of GBCA to identify pathologies, for example, tumors, that exhibit increased signal intensity. Contrast-enhanced MRI can enhance signals in specific areas of a region of interest in a subject making them stand out more clearly. For example, contrast-MRI can provide a more detailed image that can be used to, for example, determine the location, extent, and severity of an abnormality or disease. In clinical MRI, GBCAs are crucial for the detection, characterization, and monitoring of a wide area of diseases including, for example, cancer, stroke, cardiovascular disease, vascular diseases, neurodegenerative diseases, as well as chronic diseases of the kidney, liver, and pancreas. However, the use of GBCAs can present a variety of problems, for example: 1) patient discomfort during intravenous injection; 2) the need for skilled manpower, hardware, and cost; 3) longer scan times, so higher risk for motion artifacts; 4) certain patient risks (e.g., deposition of GBCAs in the brain is a safety concern); and 5) GBCAs are water pollutants which presents an environmental concern.

There is a need for systems and methods for synthesizing contrast-enhanced MR images of a subject to eliminate or reduce the need for GBCAs.

### SUMMARY OF THE DISCLOSURE

In accordance with an embodiment, a system for generating contrast-enhanced magnetic resonance images of a subject includes an input configured to receive at least one non-contrast enhanced image of the subject, and a contrast-enhanced magnetic resonance (MR) image synthesis neural network coupled to the input and configured to generate a contrast-enhanced magnetic resonance image of the subject based on the at least one non-contrast enhanced image of the subject. The contrast-enhanced MR image synthesis neural network is trained using a set of training data comprising at least quantitative data.

In accordance with another embodiment, a method for generating contrast-enhanced magnetic resonance images of a subject includes receiving, using a processor device, at least one non-contrast enhanced image of the subject, providing, using the processor device, the at least one non-contrast enhanced image to a contrast-enhanced magnetic resonance (MR) image synthesis neural network, wherein the contrast-enhanced MR image synthesis neural network is trained using a set of training data comprising at least quantitative data, generating, using the processor device, a contrast-enhanced magnetic resonance image of the subject using the contrast-enhanced MR image synthesis neural network, and displaying, using a display, the contrast-enhanced magnetic resonance image of the subject.

### BRIEF DESCRIPTION OF THE DRAWINGS

The present invention will hereafter be described with reference to the accompanying drawings, wherein like reference numerals denote like elements.

FIG. 1 is a block diagram of a system for generating contrast-enhanced magnetic resonance images of a subject in accordance with an embodiment;

FIG. 2 illustrates a method for generating contrast-enhanced magnetic resonance images of a subject in accordance with an embodiment;

FIG. 3 illustrates a method for training a neural network for generating contrast-enhanced magnetic resonance images of a subject in accordance with an embodiment;

FIG. 4 is schematic diagram of an example magnetic resonance imaging (MRI) system in accordance with an embodiment; and

FIG. 5 is a block diagram of an example computer system in accordance with an embodiment.

### DETAILED DESCRIPTION

The present disclosure describes systems and methods for generating (or synthesizing) a contrast-enhanced (e.g., a gadolinium-enhanced) magnetic resonance image of a subject from non-contrast-enhanced magnetic resonance image(s) of the subject using a contrast-enhanced MR image synthesis neural network (e.g., a deep learning neural network). The contrast-enhanced MR image synthesis neural network can be used to generate a contrast-enhanced image of the subject without the use of gadolinium-based contrast agent (GBCA), thereby mitigating associated drawbacks such as, for example, patient risks, discomfort, dependence on skilled personnel and specialized hardware, increased costs, prolonged scan times, elevated susceptibility to motion artifacts, and environmental pollution.

Advantageously, the contrast-enhanced MR image synthesis neural network can be trained using at least quantitative data such as, for example, magnetic resonance fingerprinting (MRF) data and maps, and synthetic contrast-weighted images, for example, synthetic contrast-weighted images generated from MRF data. As opposed to weighted clinical MR images (e.g., T1-weighted (T1w), T2-weighted (T2w), FLAIR (Fluid Attenuated Inversion Recovery, etc.)), which can easily lose reproducibility due to scanner and site imperfections and variations, quantitative data such as MRF maps acquire truly quantitative tissue parameters, are more robust against these imperfections, and can more accurately identify contrast-enhanced regions. By leveraging quantitative maps and synthetic images (e.g., generated from MRF data) instead of solely weighted clinical images for training of the contrast-enhanced MR image synthesis neural network, the disclosed systems and methods can provide an enhanced ability to predict or synthesize contrast-enhanced regions with heightened accuracy and robustness.

The contrast-enhanced MR image synthesis neural network can be used in clinical settings to eliminate the necessity of acquiring traditional clinical images both pre- and post-contrast. Rather, a trained contrast-enhanced MR image synthesis neural network may only require an input of one or more non-contrast images of a subject which can shorten acquisition time and costs and also ensure more reproducible results. In some embodiments, the contrast-enhanced MR image synthesis neural network can be configured to incorporate regional attention weights to enhance prediction or synthesis of the contrast-enhancement. In one example, the contrast-enhanced MR image synthesis neural network can apply additional weights to image voxels within necrotic core regions, enhancing the specificity of contrast detection. Embodiments of the contrast-enhanced MR image synthesis neural network that incorporate regional attention weights can be highly versatile, allowing adjustments such as, for example, modification with different weighting factors for various focal regions; enhancement using voxel intensity statistics, images features, or radiomics features for refined weighting; extension to a variety of types of anatomical regions and/or pathological regions of interest to further improve synthesis (or prediction) or emphasize other clinically relevant structures or information.

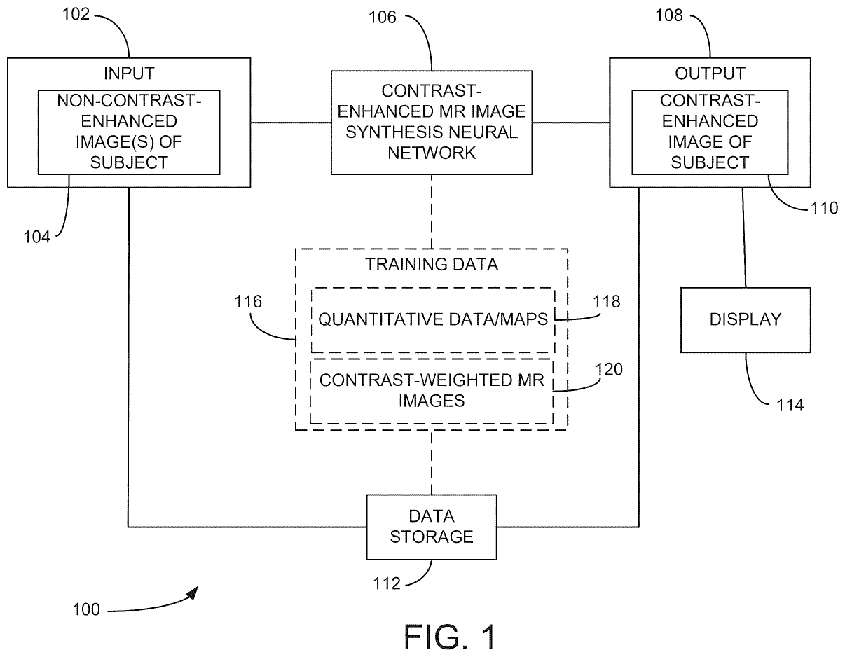

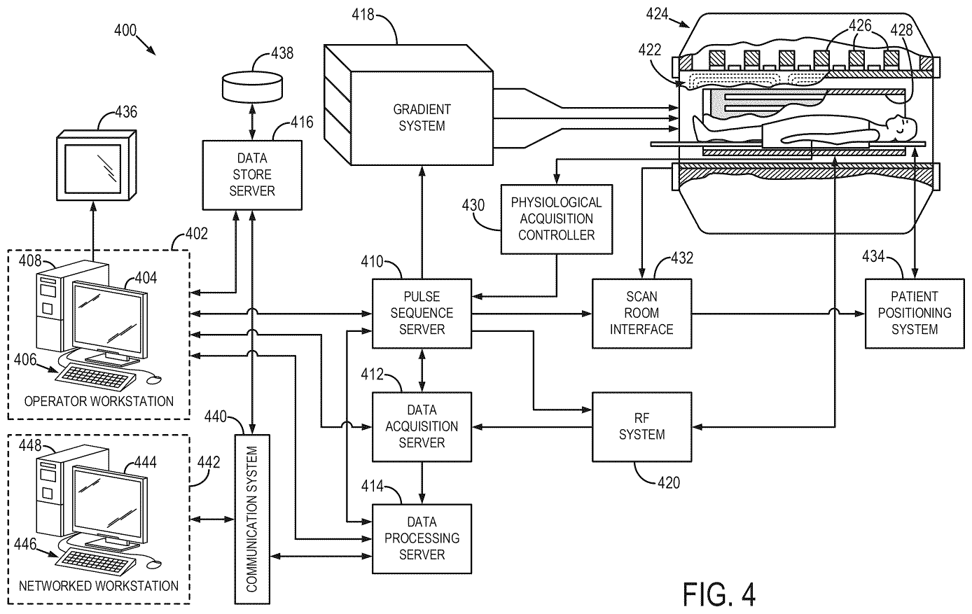

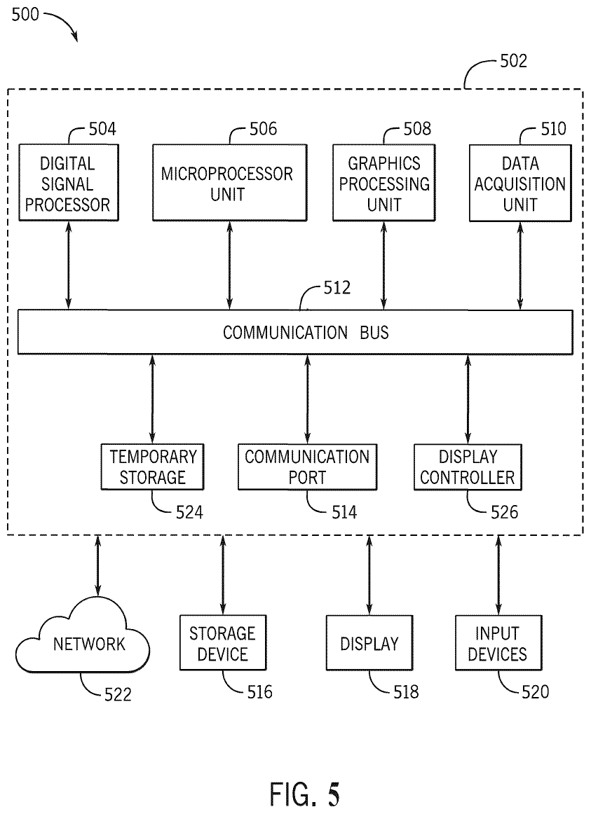

FIG. 1 is a block diagram of a system for generating contrast-enhanced magnetic resonance images of a subject in accordance with an embodiment. The system 100 can include an input 102 (e.g., one or more non-contrast-enhanced images 104 of a subject), a contrast-enhanced MR image synthesis neural network 106 (e.g., a deep learning neural network), an output 108 (e.g., a contrast-enhanced image 110 of the subject), data storage 112, and a display 114. The system 100 can be configured to generate (or synthesize) a contrast-enhanced image 110 of the subject using the contrast-enhanced MR image synthesis neural network 106. As mentioned, the input 102 can include one or more non-contrast-enhanced images 104 of the subject. The non-contrast-enhanced image(s) 104 of the subject can be acquired from the subject using an MRI system (e.g. MRI system 400 shown in FIG. 4) using known acquisition techniques and protocols. For example, the non-contrast-enhanced image or images 104 of the subject can be one or more of, for example, clinical images (e.g., contrast-weighted images), magnetic resonance fingerprinting (MRF) data, MRF maps, synthetic images (e.g., synthetic contrast-weighted images created from MRF data). In some embodiments, the non-contrast-enhanced image(s) 104 of the subject can be retrieved from data storage 112 of system 100, data storage of an MRI system used to acquire the non-contrast-enhanced image(s) 104 (e.g., disc storage 438 of MRI system 400 shown in FIG. 4). or a data storage of other computer systems (e.g., storage device 516 of a computer system 500 shown in FIG. 5). In some embodiments, the non-contrast-enhanced image(s) 104 of the subject can be acquired in real time from the subject using an MRI system (e.g., MRI system 400 shown in FIG. 4) and the system 100 may be implemented inline with a reconstruction pipeline.

The non-contrast-enhanced image(s) 104 of the subject may be provided as an input 102 to the contrast-enhanced MR image synthesis neural network 106. The non-contrast-enhanced image(s) can be provided to a contrast-enhanced MR image synthesis neural network 106 that has been trained using the same type of image and data as the image(s) 104. For example, if the non-contrast-enhanced image(s) 104 of the subject are MRF T1 and T2 maps, these image(s) 104 can be provided to a contrast-enhanced MR image synthesis neural network 106 that has been trained using MRF training data. In another example, if the non-contrast-enhanced image(s) 104 of the subject include MRF data and synthetic MR images (e.g., generated from MRF data), these image(s) 104 can be provided to a contrast-enhanced MR image synthesis neural network 106 trained using MRF and synthetic MR images. As discussed further below, a different contrast-enhanced MR image synthesis neural network 106 can be trained for various types and combinations of quantitative MR data, synthetic MR images, and contrast-weighted MR images (e.g., obtained from clinical protocols). In some embodiments, the non-contrast-enhanced image(s) 104 of the subject can be pre-processed before being input to the contrast-enhanced MR image synthesis neural network 106. The contrast-enhanced MR image synthesis neural network 106 can be trained and configured to generate (or synthesize) a contrast-enhanced image 110 of the subject from the input non-contrast-enhanced image(s) 104 of the subject. In some embodiments, the contrast-enhanced MR image synthesis neural network 106 can be a deep learning neural network that may be implemented using deep learning models or architectures. In some embodiments, the contrast-enhanced MR image synthesis neural network 106 can be a deep convolutional neural network (dCNN) that can employ, for example, a series of layers to discern features within the input non-contrast-enhanced image(s) 104. In some embodiments, the dCNN structure can be based on a U-Net architecture.

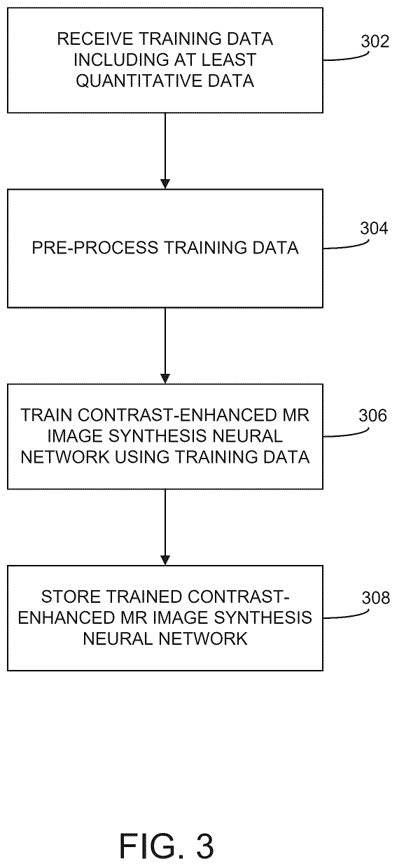

In some embodiments, the contrast-enhanced MR image synthesis neural network 106 can be trained using training data or dataset 116. As mentioned, advantageously, the training data 116 includes at least quantitative data 118 such as, for example, MRF data, MRF maps, synthetic images generated using MRF data, and/or quantitative data acquitted with other quantitative MR techniques or protocols (e.g., inversion recovery fast spin echo (IRFSE) for T1 mapping, multi-echo spin echo (MESE) and fast spin echo for T2 mapping). The training data 116 can also include clinical contrast-weighted MR images 120. The training data 116 can include existing pre- and post-contrast data and images for a plurality of subjects. The quantitative data and maps can include, for example, pre- and post-contrast MRF data and maps (e.g., T1, T1, M0). The quantitative data and maps 118 can also include synthetic contrast-weighted images (sT1w, sT2w, and sFLAIR) generated from pre- and post-contrast MRF data using known methods to synthesize contrast-weighted images from MRF data. The clinical contrast-weighted images 120 can include, for example, pre- and post-contrast T1w, T2w, FLAIR, diffusion-weighted imaging (DWI), etc., images. In some embodiments, the training data (e.g., clinical images and quantitative data) are of the same anatomy (e.g., the brain, heart, lungs, etc.). As mentioned, different combinations of training data can be used to train different contrast-enhanced MR image synthesis neural networks 106. For example, a first contrast-enhanced MR image synthesis neural network 106 can be trained using MRF data and clinical images and a second contrast-enhanced MR image synthesis neural network 106 can be trained using MRF data and synthetic images generated from MF data. In some embodiments, the contrast-enhanced MR image synthesis neural network 106 can be trained using known training methods for deep learning neural networks or models. An embodiment of a training process for the contrast-enhanced MR image synthesis neural network 106 is described below with respect to FIG. 3.

The output 108 of the contrast-enhanced MR image synthesis neural network 106 (e.g., a contrast-enhanced image 110 of the subject) may be stored in, for example, data storage 112 of the system 100, data storage of an MRI system (e.g., MRI system 400 shown in FIG. 4), or data storage of other computer system s (e.g., storage device 516 of computer system 500 shown in FIG. 5. The contrast-enhanced image 110 of the subject may also be displayed to a user or operator on a display 114 (e.g., display 404 of an MRI system 400 shown in FIG. 4, or a display 518 of a computer system 500 shown in FIG. 5).

In some embodiments, the contrast-enhanced MR image synthesis neural network 106 may be implemented on one or more processors (or processor devices) of a computer system such as, for example, any general purpose computer system or device, such as a personal computer, workstation, cellular phone, smartphone, laptop, tablet, or the like. As such, the computer system may include any suitable hardware and components designed or capable of carrying out a variety of processing and control tasks, including, for example, steps for implementing the contrast-enhanced MR image synthesis neural network 106, and receiving non-contrast-enhanced image(s) 104 of the subject. For example, the computer system may include a programmable processor or combination of programmable processors, such as central processing units (CPUs), graphics processing units (GPUs), and the like. In some implementations, the one or more processors of the computer system may be configured to execute instructions stored in a non-transitory computer readable-media. In this regard, the computer system may be any device or system designed to integrate a variety of software, hardware, capabilities and functionalities. Alternatively, and by way of particular configurations and programming, the computer system may be a special-purpose system or device. For instance, such special-purpose system or device may include one or more dedicated processing units or modules that may be configured (e.g., hardwired, or pre-programmed) to carry out steps, in accordance with aspects of the present disclosure.

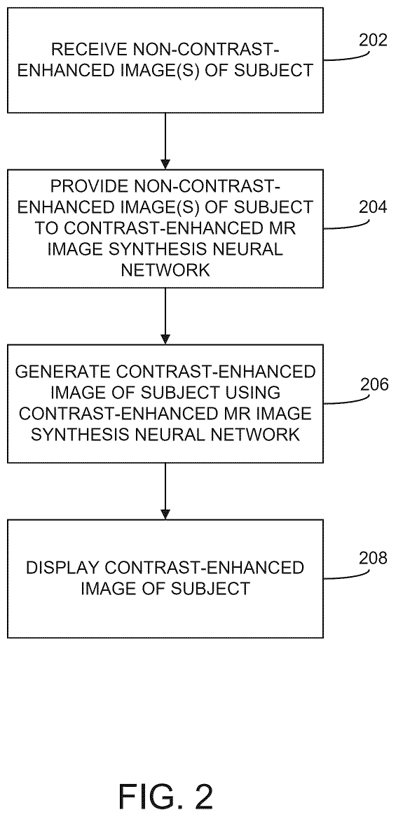

FIG. 2 illustrates a method for generating contrast-enhanced magnetic resonance images of a subject in accordance with an embodiment. The process illustrated in FIG. 2 is described below as being carried out by the system 100 for generating a contrast-enhanced image of a subject as illustrated in FIG. 1, however, in some examples, the process of FIG. 2 may be implemented by another system. Although the blocks of the process are illustrated in a particular order, in some embodiments, one or more blocks may be executed in a different order than illustrated in FIG. 2 or may be bypassed. The process of FIG. 2 may be performed by a processing system including at least one electronic processor, where the at least one electronic processor may be or include a processor as described herein (e.g., including one or more individual processor devices) with respect to FIGS. 1, 4 and 5. For example, the process of FIG. 2 may be performed using a processor of MRI system 400 (e.g., processor 408 of a workstation 402) described below with respect to FIG. 4 or computer system 500 described below with respect to FIG. 5.

At block 202, one or more non-contrast-enhanced image(s) 104 of a subject may be received. The non-contrast-enhanced image(s) 104 can be acquired from the subject using an MRI system (e.g., MRI system 400 shown in FIG. 4) using known acquisition techniques and protocols. As mentioned, in some embodiments, the non-contrast-enhanced image or images 104 of the subject can be one or more of, for example, clinical images (e.g., contrast-weighted images), magnetic resonance fingerprinting (MRF) data, MRF maps, synthetic images (e.g., synthetic contrast-weighted images created from MRF data). In some embodiments, the non-contrast-enhanced image(s) 104 can be retrieved from data storage 112 of system 100, data storage of an MRI system used to acquire the non-contrast-enhanced image(s) 104, or data storage of other computer systems (e.g., storage device 516 of computer system 500 shown in FIG. 5).

At block 204, the non-contrast-enhanced image(s) 104 of the subject can be provided as an input to a trained contrast-enhanced MR image synthesis neural network 106. As mentioned, the trained contrast-enhanced MR image synthesis neural network 106 can be a network trained using the particular type of non-contrast-enhanced image(s) of the subject. For example, if the non-contrast-enhanced image(s) 104 of the subject are MRF T1 and T2 maps, these image(s) 104 can be provided to a contrast-enhanced MR image synthesis neural network 106 that has been trained using MRF training data. The contrast-enhanced MR image synthesis neural network 106 may be a deep learning neural network and implemented using deep learning models or architectures. At block, 206, the contrast-enhanced MR image synthesis neural network 106 can be used to generate (or synthesize) a contrast-enhanced image 110 of the subject from the input non-contrast-enhanced image(s) 104 of the subject. At block 208, the generated (or synthesized) contrast-enhanced image 110 of the subject can be displayed on a display 114 (e.g., a display of an MRI system (e.g., MRI system 400 shown in FIG. 4), display 518 of computer system 500 shown in FIG. 5). In some embodiments, the generated contrast-enhanced image 110 of the subject can be stored in data storage 112.

As mentioned, the contrast-enhanced MR image synthesis neural network 106 (shown in FIG. 1) can be trained to generate a contrast-enhanced image 110 (shown in FIG. 1) of the subject using a set of training data 116 (shown in FIG. 1). FIG. 3 illustrates a method for training a neural network for generating contrast-enhanced magnetic resonance images in accordance with an embodiment. The process illustrated in FIG. 3 is described below as being carried out by the system 100 for generating a contrast-enhanced image of a subject as illustrated in FIG. 1, however, in some examples, the process of FIG. 3 may be implemented by another system. Although the blocks of the process are illustrated in a particular order, in some embodiments, one or more blocks may be executed in a different order than illustrated in FIG. 3 or may be bypassed. The process of FIG. 3 may be performed by a processing system including at least one electronic processor, where the at least one electronic processor may be or include a processor as described herein (e.g., including one or more individual processor devices) with respect to FIGS. 1, 4 and 5. For example, the process of FIG. 3 may be performed using a processor of MRI system 400 (e.g., processor 408 of a workstation 402) described below with respect to FIG. 4 or computer system 500 described below with respect to FIG. 5.

At block 302, training data 116 may be received. As mentioned, the training data advantageously includes at least quantitative data or maps 118, for example, MRF data, and MRF maps (e.g., T1, T2, M0). In some embodiments, the quantitative data and maps can include simulated contrast-weighted images, for example, simulated from MRF data. In some embodiments, the training data 116 can also include clinical contrast-weighted images 120, for example, T1w, T2w, FLAIR, DWI, etc. The training data 116 can include existing pre- and post-contrast quantitative data and images for a plurality of subjects. In some embodiments, the training data 116 may be retrieved from data storage (or memory), for example, data storage 112 of system 100 shown in FIG. 1, or data storage of other computer systems (e.g., storage device 516 of computer system 500 shown in FIG. 5). At block 304, the training data can be preprocessed. In some embodiments, for example, the input training data 116 can be divided into training image patches and then fed into the contrast-enhanced MR image synthesis neural network 106. In addition, in some embodiments, the training data 116 can be registered, skull stripped, or intensity normalized using known methods before being input into the contrast-enhanced MR image synthesis neural network 106.

At block 306, the contrast-enhanced MR image synthesis neural network 106 can be trained for generating (or synthesizing) a contrast-enhanced image of the subject using the training data. In some embodiments, for example, the training image patches can be provided to the contrast-enhanced MR image synthesis neural network 106 to extract feature maps. In some embodiments, the contrast-enhanced MR image synthesis neural network 106 can employ a convolutional layer to map the extracted features and generate the output synthesized (or generated) contrast-enhanced image 110 (e.g., a gadolinium T1-weighted (Gd-T1w) image). In some embodiments, the contrast-enhanced images generated by the contrast-enhanced MR image synthesis neural network 106 based on the training data can be compared to ground truth contrast-enhanced images from the training data 116 (e.g., associated with the plurality of subjects on the training data 116), for example, using a loss function. Parameters, including, for example, kernel size, input patch size, and the number of input images can be modified, for example, by a user via a user input (e.g., input devices 520 of computer system 500 shown in FIG. 5). In some embodiments, the contrast-enhanced MR image synthesis neural network 106 can incorporate regional attention weights to enhance the synthesis or predictions of the contrast-enhancement, for example, to selectively encourage model training/improvement in hard-to-synthesize areas of focal enhancement. In one example, the contrast-enhanced MR image synthesis neural network 106 can be configured to apply additional weights to image voxels within necrotic core regions, enhancing the specificity of contrast detection, for example, voxels belonging to the necrotic core can be scaled by a predetermined factor of 2 to enhance model sensitivity to necrotic regions, and improve tumor prediction accuracy. In some embodiments, a region of interest (ROI) mask (e.g., a regional segmentation) for the application of regional attention weights can be retrieved from data storage 112. The ROI mask can be provided to the contrast-enhanced MR image synthesis neural network 106 during training, for example, as part of the training data 116.

In some embodiments, the contrast-enhanced MR image synthesis neural network 106 can be trained using known training methods for beep learning neural networks or models. The contrast-enhanced MR image synthesis neural network 106 can be, for example, an artificial neural network (e.g., a convolutional neural network (CNN), a generative adversarial network (GAN), transformers, etc. The contrast-enhanced MR image synthesis neural network 106 can be trained using known methods such as, for example, supervised learning, self-supervised learning, semi-supervised learning, etc. As one example, to perform supervised learning, the training data includes example inputs and corresponding desired (for example, actual) outputs, and the machine learning model progressively develops a model that maps inputs to the outputs included in the training data. As another example, to perform self-supervised learning, a model is trained on a task using the data itself to generate supervisory signals (e.g., unlabeled training data), rather than relying on, e.g., external labels provided by a user (e.g., labeled training data). As yet another example, to perform semi-supervised learning, the training data may include desired output values for a subset of the training data (e.g., labeled training data) while the remaining training data may be unlabeled or imprecisely labeled (e.g., unlabeled training data). As mentioned, the contrast-enhanced MR image synthesis neural network 106 can be trained using one or more processor devices. At block 308, the trained contrast-enhanced MR image synthesis neural network 106 can be stored in data storage (e.g., data storage 112 of system 100, data storage of a computer system such as storage device 516 of computer system 500 shown in FIG. 5).

FIG. 4 shows an example of an MRI system 400 that may be used to implement the methods described herein. MRI system 400 includes an operator workstation 402, which may include a display 404, one or more input devices 406 (e.g., a keyboard, a mouse), and a processor 408. The processor 408 may include a commercially available programmable machine running a commercially available operating system. The operator workstation 402 provides an operator interface that facilitates entering scan parameters into the MRI system 400. The operator workstation 402 may be coupled to different servers, including, for example, a pulse sequence server 410, a data acquisition server 412, a data processing server 414, and a data store server 416. The operator workstation 402 and the servers 410, 412, 414, and 416 may be connected via a communication system 440, which may include wired or wireless network connections.

The pulse sequence server 410 functions in response to instructions provided by the operator workstation 402 to operate a gradient system 418 and a radiofrequency (“RF”) system 420. Gradient waveforms for performing a prescribed scan are produced and applied to the gradient system 418, which then excites gradient coils in an assembly 422 to produce the magnetic field gradients Gₓ, G_{y}, and G_{z} that are used for spatially encoding magnetic resonance signals. The gradient coil assembly 422 forms part of a magnet assembly 424 that includes a polarizing magnet 426 and a whole-body RF coil 428.

RF waveforms are applied by the RF system 420 to the RF coil 428, or a separate local coil to perform the prescribed magnetic resonance pulse sequence. Responsive magnetic resonance signals detected by the RF coil 428, or a separate local coil, are received by the RF system 420. The responsive magnetic resonance signals may be amplified, demodulated, filtered, and digitized under direction of commands produced by the pulse sequence server 410. The RF system 420 includes an RF transmitter for producing a wide variety of RF pulses used in MRI pulse sequences. The RF transmitter is responsive to the prescribed scan and direction from the pulse sequence server 410 to produce RF pulses of the desired frequency, phase, and pulse amplitude waveform. The generated RF pulses may be applied to the whole-body RF coil 428 or to one or more local coils or coil arrays.

The RF system 120 also includes one or more RF receiver channels. An RF receiver channel includes an RF preamplifier that amplifies the magnetic resonance signal received by the coil 428 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

$$M = \sqrt{{I}^{2}} + {Q}^{2} \qquad ( 1 )$$

and the phase of the received magnetic resonance signal may also be determined according to the following relationship:

$$φ = {tan}^{- 1} ( \frac{Q}{I} ) \qquad ( 2 )$$

The pulse sequence server 410 may receive patient data from a physiological acquisition controller 430. By way of example, the physiological acquisition controller 430 may receive signals from a number of different sensors connected to the patient, including electrocardiograph (“ECG”) signals from electrodes, or respiratory signals from a respiratory bellows or other respiratory monitoring devices. These signals may be used by the pulse sequence server 410 to synchronize, or “gate,” the performance of the scan with the subject's heartbeat or respiration.

The pulse sequence server 410 may also connect to a scan room interface circuit 432 that receives signals from various sensors associated with the condition of the patient and the magnet system. Through the scan room interface circuit 432, a patient positioning system 434 can receive commands to move the patient to desired positions during the scan.

The digitized magnetic resonance signal samples produced by the RF system 420 are received by the data acquisition server 412. The data acquisition server 412 operates in response to instructions downloaded from the operator workstation 402 to receive the real-time magnetic resonance data and provide buffer storage, so that data is not lost by data overrun. In some scans, the data acquisition server 412 passes the acquired magnetic resonance data to the data processor server 414. In scans that require information derived from acquired magnetic resonance data to control the further performance of the scan, the data acquisition server 412 may be programmed to produce such information and convey it to the pulse sequence server 410. For example, during pre-scans, magnetic resonance data may be acquired and used to calibrate the pulse sequence performed by the pulse sequence server 410. As another example, navigator signals may be acquired and used to adjust the operating parameters of the RF system 420 or the gradient system 418, or to control the view order in which k-space is sampled. In still another example, the data acquisition server 412 may also process magnetic resonance signals used to detect the arrival of a contrast agent in a magnetic resonance angiography (“MRA”) scan. For example, the data acquisition server 412 may acquire magnetic resonance data and processes it in real-time to produce information that is used to control the scan.

The data processing server 414 receives magnetic resonance data from the data acquisition server 412 and processes the magnetic resonance data in accordance with instructions provided by the operator workstation 402. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images.

Images reconstructed by the data processing server 414 are conveyed back to the operator workstation 402 for storage. Real-time images may be stored in a database memory cache, from which they may be output to operator display 402 or a display 436. Batch mode images or selected real time images may be stored in a host database on disc storage 438. When such images have been reconstructed and transferred to storage, the data processing server 414 may notify the data store server 416 on the operator workstation 402. The operator workstation 402 may be used by an operator to archive the images, produce films, or send the images via a network to other facilities.

The MRI system 400 may also include one or more networked workstations 442. For example, a networked workstation 442 may include a display 444, one or more input devices 446 (e.g., a keyboard, a mouse), and a processor 448. The networked workstation 442 may be located within the same facility as the operator workstation 402, or in a different facility, such as a different healthcare institution or clinic.

The networked workstation 442 may gain remote access to the data processing server 414 or data store server 416 via the communication system 440. Accordingly, multiple networked workstations 442 may have access to the data processing server 414 and the data store server 416. In this manner, magnetic resonance data, reconstructed images, or other data may be exchanged between the data processing server 414 or the data store server 416 and the networked workstations 442, such that the data or images may be remotely processed by a networked workstation 442.

As mentioned above, the quantitative data 118 (shown in FIG. 1) used for training the contrast-enhanced MR image synthesis neural network 106 (shown in FIG. 1) can include, for example, MRF data and maps. MRF is a technique that facilitates mapping of tissue or other material properties based on random or pseudorandom measurements of the subject or object being imaged. In particular, MRF can be conceptualized as evolutions in different “resonant species” to which the RF is applied. The term “resonant species,” as used herein, refers to a material, such as water, fat, bone, muscle, soft tissue, and the like, that can be made to resonate using NMR. By way of illustration, when radio frequency (“RF”) energy is applied to a volume that has both bone and muscle tissue, then both the bone and muscle tissue will produce a nuclear magnetic resonance (“NMR”) signal; however, the “bone signal” represents a first resonant species and the “muscle signal” represents a second resonant species, and thus the two signals will be different. These different signals from different species can be collected simultaneously over a period of time to collect an overall “signal evolution” for the volume.

The measurements obtained in MRF techniques are achieved by varying the acquisition parameters from one repetition time (“TR”) period to the next, which creates a time series of signals with varying contrast. Examples of acquisition parameters that can be varied include flip angle (“FA”), RF pulse phase, TR, echo time (“TE’), and sampling patterns, such as by modifying one or more readout encoding gradients. The acquisition parameters are varied in a random manner, pseudorandom manner, or other manner that results in signals from different materials or tissues to be spatially incoherent, temporally incoherent, or both. For example, in some instances, the acquisition parameters can be varied according to a non-random or non-pseudorandom pattern that otherwise results in signals from different materials or tissues to be spatially incoherent, temporally incoherent, or both.

From these measurements, which as mentioned above may be random or pseudorandom, or may contain signals from different materials or tissues that are spatially incoherent, temporally incoherent, or both, MRF processes can be designed to map any of a wide variety of parameters or properties. Examples of such parameters or properties that can be mapped may include, but are not limited to, tissue parameters or properties such as longitudinal relaxation time (T₁), transverse relaxation time (T₂), and proton density (ρ), and device dependent parameters such as main or static magnetic field map (B₀). MRF is generally described in U.S. Pat. No. 8,723,518 and Published U.S. Patent Application No. 2015/0301141, each of which is incorporated herein by reference in its entirety.

The data acquired with MRF techniques are compared with a dictionary of signal models, or templates, that have been generated for different acquisition parameters from magnetic resonance signal models, such as Bloch equation-based physics simulations. This comparison allows estimation of the physical properties, such as those mentioned above. As an example, the comparison of the acquired signals to a dictionary can be performed using any suitable matching or pattern recognition technique. The properties for the tissue or other material in a given voxel are estimated to be the values that provide the best signal template matching. For instance, the comparison of the acquired data with the dictionary can result in the selection of a signal vector, which may constitute a weighted combination of signal vectors, from the dictionary that best corresponds to the observed signal evolution. The selected signal vector includes values for multiple different quantitative properties, which can be extracted from the selected signal vector and used to generate the relevant quantitative property maps.

The stored signals and information derived from reference signal evolutions may be associated with a potentially very large data space. The data space for signal evolutions can be partially described by:

$$SE = {\sum }_{s = 1}^{{N}_{s}} {\prod }_{i = 1}^{{N}_{A}} {\sum }_{j = 1}^{{N}_{RF}} {R}_{i} ( α ) {R}_{{RF}_{ij}} ( α , ϕ ) R ( G ) {E}_{i} ( {T}_{1} , {T}_{2} , D ) {M}_{0} \qquad ( 3 )$$

where SE is a signal evolution; Nₛ is a number of spins; N_{A} is a number of sequence blocks; N_{RF} is a number of RF pulses in a sequence block; α is a flip angle; φ is a phase angle; Rᵢ(α) is a rotation due to off resonance; R_{RF}_{ᵢⱼ}(α, φ) is a rotation due to RF differences; R(G) is a rotation due to a magnetic field gradient; This a longitudinal, or spin-lattice, relaxation time; T₂ is a transverse, or spin-spin, relaxation time; D is diffusion relaxation; Eᵢ(T₁, T₂, D) is a signal decay due to relaxation differences; and M₀ is the magnetization in the default or natural alignment to which spins align when placed in the main magnetic field.

While Eᵢ(T₁, T₂, D) is provided as an example, in different situations, the decay term, Eᵢ(T₁, T₂, D), may also include additional terms, Eᵢ(T₁, T₂, D, . . . ) or may include fewer terms, such as by not including the diffusion relaxation, as Eᵢ(T₁, T₂) or Eᵢ(T₁, T₂, . . . ). Also, the summation on “j” could be replace by a product on “j”. The dictionary may store signals described by,

$${S}_{i} = {R}_{i} {R}_{i} ( {S}_{i - 1} ) \qquad ( 4 )$$

where S₀ is the default, or equilibrium, magnetization; Sᵢ is a vector that represents the different components of magnetization, Mₓ, M_{y}, and M_{z} during the i^{th} acquisition block; Rᵢ is a combination of rotational effects that occur during the i^{th} acquisition block; and Eᵢ is a combination of effects that alter the amount of magnetization in the different states for the i^{th} acquisition block. In this situation, the signal at the i^{th} acquisition block is a function of the previous signal at acquisition block (i.e., the (i−1)^{th} acquisition block). Additionally or alternatively, the dictionary may store signals as a function of the current relaxation and rotation effects and of previous acquisitions. Additionally or alternatively, the dictionary may store signals such that voxels have multiple resonant species or spins, and the effects may be different for every spin within a voxel. Further still, the dictionary may store signals such that voxels may have multiple resonant species or spins, and the effects may be different for spins within a voxel, and thus the signal may be a function of the effects and the previous acquisition blocks.

Thus, in MRF, a unique signal timecourse is generated for each pixel. This timecourse evolves based on both physiological tissue properties such as T1 or T2 as well as acquisition parameters like flip angle (FA) and repetition time (TR). This signal timecourse can, thus, be referred to as a signal evolution and each pixel can be matched to an entry in the dictionary, which is a collection of possible signal evolutions or timecourses calculated using a range of possible tissue property values and knowledge of the quantum physics that govern the signal evolution. Upon matching the measured signal evolution/timecourse to a specific dictionary entry, the tissue properties corresponding to that dictionary entry can be identified. A fundamental criterion in MRF is that spatial incoherence be maintained to help separate signals that are mixed due to undersampling. In other words, signals from various locations should differ from each other, in order to be able to separate them when aliased. As mentioned above, to achieve an MRF process, a magnetic resonance imaging (MRI) system (e.g., MRI system 400 shown in FIG. 4) may be utilized.

FIG. 5 is a block diagram of an example computer system in accordance with an embodiment. Computer system 500 may be used to implement the systems and methods described herein. In some embodiments, the computer system 500 may be a workstation, a notebook computer, a tablet device, a mobile device, a multimedia device, a network server, a mainframe, one or more controllers, one or more microcontrollers, or any other general-purpose or application-specific computing device. The computer system 500 may operate autonomously or semi-autonomously, or may read executable software instructions from the memory or storage device 516 or a computer-readable medium (e.g., a hard drive, a CD-ROM, flash memory), or may receive instructions via the input device 520 from a user, or any other source logically connected to a computer or device, such as another networked computer or server. Thus, in some embodiments, the computer system 500 can also include any suitable device for reading computer-readable storage media.

Data, such as data acquired with, for example, an imaging system (e.g., a magnetic resonance imaging (MRI) system, etc.), may be provided to the computer system 500 from a data storage device 516, and these data are received in a processing unit 502. In some embodiments, the processing unit 502 included one or more processors. For example, the processing unit 502 may include one or more of a digital signal processor (DSP) 504, a microprocessor unit (MPU) 506, and a graphic processing unit (GPU) 508. The processing unit 502 also includes a data acquisition unit 510 that is configured to electronically receive data to be processed. The DSP 504, MPU 506, GPU 508, and data acquisition unit 510 are all coupled to a communication bus 512. The communication bus 512 may be, for example, a group of wires, or a hardware used for switching data between the peripherals or between any component in the processing unit 502.

The processing unit 502 may also include a communication port 514 in electronic communication with other devices, which may include a storage device 516, a display 518, and one or more input devices 520. Examples of an input device 520 include, but are not limited to, a keyboard, a mouse, and a touch screen through which a user can provide an input. The storage device 516 may be configured to store data, which may include data such as, for example, MR data, MR images, quantitative maps, synthesized images, quantitative parameters, segmented images, etc., whether these data are provided to, or processed by, the processing unit 502. The display 518 may be used to display images, reports, and other information, such as patient health data, and so on.

The processing unit 502 can also be in electronic communication with a network 522 to transmit and receive data and other information. The communication port 514 can also be coupled to the processing unit 502 through a switched central resource, for example the communication bus 512. The processing unit 502 can also include temporary storage 524 and a display controller 526. The temporary storage 524 is configured to store temporary information. For example, the temporary storage 524 can be a random-access memory.

Computer-executable instructions for generating contrast-enhanced magnetic resonance images of a subject according to the above-described methods may be stored on a form of computer readable media. Computer readable media includes volatile and nonvolatile, removable, and non-removable media implemented in any method or technology for storage of information such as computer readable instructions, data structures, program modules or other data. Computer readable media includes, but is not limited to, random access memory (RAM), read-only memory (ROM), electrically erasable programmable ROM (EEPROM), flash memory or other memory technology, compact disk ROM (CD-ROM), digital volatile disks (DVD) or other optical storage, magnetic cassettes, magnetic tape, magnetic disk storage or other magnetic storage devices, or any other medium which can be used to store the desired instructions and which may be accessed by a system (e.g., a computer), including by internet or other computer network form of access.

The present invention has been described in terms of one or more preferred embodiments, and it should be appreciated that many equivalents, alternatives, variations, and modifications, aside from those expressly stated, are possible and within the scope of the invention.

1. A system for generating contrast-enhanced magnetic resonance images of a subject, the system comprising:
an input configured to receive at least one non-contrast enhanced image of the subject; and
a contrast-enhanced magnetic resonance (MR) image synthesis neural network coupled to the input and configured to generate a contrast-enhanced magnetic resonance image of the subject based on the at least one non-contrast enhanced image of the subject, wherein the contrast-enhanced MR image synthesis neural network is trained using a set of training data comprising at least quantitative data.

2. The system according to claim 1, wherein the quantitative data comprises one or more of magnetic resonance fingerprinting maps, magnetic resonance fingerprinting data, or synthetic images generated from magnetic resonance fingerprinting data.

3. The system according to claim 1, wherein the set of training data further comprises contrast-weighted magnetic resonance images.

4. The system according to claim 1, wherein the contrast-enhanced MR image synthesis neural network is a deep convolutional neural network (dCNN).

5. The system according to claim 1, wherein the contrast-enhanced MR image synthesis neural network is configured to include regional attention weights.

6. The system according to claim 2, wherein the magnetic resonance fingerprinting maps comprise T1, T2 and M0 maps.

7. The system according to claim 1, further comprising a display coupled to the contrast-enhanced MR image synthesis neural network and configured to display the contrast-enhanced image of the subject.

8. A method for generating contrast-enhanced magnetic resonance images of a subject, the method comprising:
receiving, using a processor device, at least one non-contrast enhanced image of the subject;
providing, using the processor device, the at least one non-contrast enhanced image to a contrast-enhanced magnetic resonance (MR) image synthesis neural network, wherein the contrast-enhanced MR image synthesis neural network is trained using a set of training data comprising at least quantitative data;
generating, using the processor device, a contrast-enhanced magnetic resonance image of the subject using the contrast-enhanced MR image synthesis neural network; and
displaying, using a display, the contrast-enhanced magnetic resonance image of the subject.

9. The method according to claim 8, wherein the quantitative data comprises one or more of magnetic resonance fingerprinting maps, magnetic resonance fingerprinting data, or synthetic images generated from magnetic resonance fingerprinting data.

10. The method according to claim 9, wherein the magnetic resonance fingerprinting maps comprise T1, T2 and M0 maps.

11. The method according to claim 8, wherein the set of training data further comprises contrast-weighted magnetic resonance images.

12. The method according to claim 1, wherein the contrast-enhanced MR image synthesis neural network is a deep convolutional neural network (dCNN).

13. The method according to claim 8, wherein the contrast-enhanced MR image synthesis neural network is configured to include regional attention weights.

14. The method according to claim 8, wherein the set of training data comprises pre-contrast and post-contrast training data.
